(19) United States
(12) Patent Application Publication
(10) Pub. No.: US 2007/0218459 A1
(43) Pub. Date: Sep. 20, 2007

(54) Diagnostic System For Otolaryngologic Pathogens And Use Thereof

(76) Inventors: Benjamin L. Miller, Penfield, NY (US); Scott R. Horner, Rochester, NY (US); Lewis J. Rothberg, Pittsford, NY (US); Farhan Taghizadeh, Albuquerque, NM (US)

Correspondence Address: NIXON PEABODY LLP - PATENT GROUP, CLINTON SQUARE, P.O. BOX 31051, ROCHESTER, NY, 14603-1051, US

(21) Appl. No.: 10/572,080

(22) PCT Filed: Sep. 20, 2004
(86) PCT No.: PCT/US04/30644

§ 371 Date: May 11, 2007

Related U.S. Application Data
(60) Provisional application No. 60/504,530, filed on Sep. 19, 2003.

(51) Int. Cl. C12Q 1/68; C07H 21/04

(52) U.S. Cl. 435/6; 536/24.32

(57) ABSTRACT

The present invention relates to a method of detecting the presence of an otolaryngologic pathogen in a biological sample, and a sensor device, sensor chip, and nucleic acid probes useful for detecting otolaryngologic pathogens.

This application claims the benefit of U.S. Provisional Patent Application Ser. No. 60/504,530, filed Sep. 19, 2003, which is hereby incorporated by reference in its entirety.

The present invention was made, at least in part, with funding received from the U.S. Department of Energy under grant DE-FG02-02ER63410.A000: The U.S. government may retain certain rights in this invention.

### FIELD OF THE INVENTION

The present invention relates to diagnostic systems for common otolaryngologic pathogens and nucleic acid probes used therein.

### BACKGROUND OF THE INVENTION

Point of care diagnosis of infectious organisms would dramatically change treatment paradigms in otolaryngologic disease. For example, the prevalent spread of bacterial antibiotic resistance could be slowed if better diagnostic capabilities existed at the point of care (Sinus and Allergy Health Partnership, “Antimicrobial Treatment for Acute Bacterial Rhinosinusitis,” Otolaryngology-Head and Neck Surgery, 123-1:S12 Figure 6 (2000)). Additionally, such testing capabilities could reduce the cost of care, better enabling the correlation of symptoms and clinical findings to the presence of infectious organisms. Such point of care technologies are widespread in modern medical care, from blood glucose measurements to rapid Group A Streptococcus testing. Acceptability of basic rapid testing as well as its many benefits has prompted research to find wider uses for this technology in otolaryngology.

Bacterial and viral species identification using comparative analysis of rDNA sequences is a well established method of bacterial identification (Ludwig et al., “Phylogeny of Bacteria Beyond the 16S rRNA Standard,” ASM News, 65:752-757 (1999)). Recent advances in targeting ribosomal nucleic acid sequences (rRNA) with DNA (rDNA) probes represents an attractive technique for rapid detection without sequence amplification, given the abundance of such ribosomes in bacteria (Trotha et al., “Rapid Ribosequencing—An Effective Diagnostic Tool for Detecting Microbial Infection” Infection, 29:12-16 (2001); Knut et al., “Development and Evaluation of a 16S Ribosomal DNA Array-Based Approach for Describing Complex Microbial Communities in Ready-To-Eat Vegetable Salads Packed in a Modified Atmosphere,” Applied and Environmental Microbiology, 68:1146-1156 (2002)). Using sequence databases, bacteria specific sequences have been identified, with sequences for Pseudomonas proving reasonably sensitive for detection (Perry-O'Keefe et al., “Identification of Indicator Microorganisms Using A Standardized PNA FISH Method,” J. Microbiol. Meth., 47:281-292 (2001)). Pseudomonas aeruginosa represents an excellent organism for early biosensor development in otolaryngology not only because of its pathogenicity in ear infections like otitis externa, but also because of its presence in normal ears (Roland et al., “Mcrobiology of Acute Otitis Externa” The Laryngoscope, 112:1166-1177 (2002)). Detection research must be geared towards providing accurate counts of such organisms in the clinical setting.

The present invention is directed to overcoming these and other deficiencies in the art.

### SUMMARY OF THE INVENTION

A first aspect of the present invention relates to a method of detecting the presence of an otolaryngologic pathogen in a biological sample. This method involves providing a sensor device including (i) a substrate having two or more nucleic acid probes respectively confined to two or more distinct locations thereon, and (ii) a detector that detects the binding of target nucleic acids to the two or more nucleic acid probes, wherein a target nucleic acid is specific to one or more otolaryngologic pathogens; exposing a biological sample, or a portion thereof, to the sensor device under conditions effective to allow hybridization between the two or more nucleic acid probes and a target nucleic acid to occur; and detecting with the detector whether any target nucleic acid hybridizes to the two or more nucleic acid probes, where hybridization indicates the presence of the otolaryngologic pathogen in the biological sample and presence of more than one otolaryngologic pathogen can be detected simultaneously.

A second aspect of the present invention relates to a sensor device that includes a substrate having two or more nucleic acid probes respectively confined to two or more distinct locations thereon, and a detector that detects the hybridization of target nucleic acids to the two or more nucleic acid probes upon exposure to a biological sample, wherein a target nucleic acid is specific to one or more otolaryngologic pathogens and hybridization indicates presence of the otolaryngologic pathogen in the biological sample, the detector being capable of simultaneously detecting presence of more than one otolaryngologic pathogen in the biological sample.

A third aspect of the present invention relates to a sensor chip that includes a substrate having two or more nucleic acid probes respectively confined to two or more distinct locations thereon, the nucleic acid probes hybridizing to a target nucleic acid of an otolaryngologic pathogen under suitable hybridization conditions, wherein the two or more probes are selected to hybridize, collectively, to target nucleic acids of two or more otolaryngologic pathogens.

A fourth aspect of the present invention relates to a nucleic acid probe having a nucleic acid sequence selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, complements thereof, and combinations thereof.

The present invention is meant to broaden the capabilities for point-of-care infection detection, allowing for the rapid diagnosis of many common bacterial, viral, and fungal infections, particularly as they relate to otolaryngologic pathogens.

### BRIEF DESCRIPTION OF THE DRAWINGS

FIG. 1 is a schematic diagram of a nanocrystal sensor chip that includes a nucleic acid probe attached to a nanocrystal particle, and a second non-target nucleic acid attached to a quenching agent that quenches, absorbs, or shifts fluorescence of the nanoparticle. In the absence of a target nucleic acid molecule, the quenching agent prevents detection of nanocrystal fluorescence. In the presence of the target nucleic acid, which has a greater affinity for the target than the non-target does, the non-target nucleic acid is displaced, and fluorescence can be detected.

FIG. 2 illustrates schematically a nanocrystal sensor device of the present invention which includes, as a component thereof, a nanocrystal sensor chip of the present invention.

FIG. 3 illustrates schematically a porous semiconductor (Si) structure for use in a microcavity sensor chip. A porous silicon structure is shown, with the enlargement showing an electron micrograph image of the central layer. Etched pores within the central layer are clearly visible. This porous semiconductor chip can be used to replace the chip shown in FIG. 2.

FIG. 4 illustrates an interferometric chip for use in an interferometric sensor device of the present invention.

FIG. 5 illustrates an interferometric sensor device in accordance with one embodiment of the present invention.

FIG. 6 illustrates schematically a nucleic acid hairpin sensor chip of the present invention. A hairpin nucleic acid probe is immobilized at one end thereof to a fluorescent quenching surface, and the other end thereof has attached thereto a fluorophore. In the hairpin conformation, the fluorophore is in sufficiently close proximity to the fluorescent quenching surface such that fluorescent emissions of the fluorophore are quenched. In the presence of a target nucleic acid molecule, the hairpin conformation is lost, resulting in detectable fluorescent emissions. This hairpin sensor chip can be used to replace the chip shown in FIG. 2.

FIG. 7 illustrates schematically a microfluidic chip of the present invention. A microfluidic chip is constructed to contain one reservoir (A) containing a solution of the quenched fluorescent probe, a fill port (B) into which the sample is introduced, and a visualization chamber (C), which can be probed with a spectrophotometer. The sample to be analyzed is introduced into (B), and then fluidic flow is induced to mix the contents of (A) and (B) in the channel, bringing the mixed solution to (C). If the target DNA sequence is present, unquenching of the fluorescent probe occurs (or, alternatively, a color change occurs based on interaction/lack of interaction with Au nanoparticles), and the signal may be read spectrophotometrically through (C).

FIG. 8 is a schematic diagram illustrating the chemical coating of the biosensor.

FIG. 9 is a schematic diagram showing the placement of the probes on the chip in the probe testing experiment. The probes were placed on one side (left), and the probe and its complementary sequence on the other (right).

FIG. 10 is a schematic diagram showing the optical scanning of Probe 1 (right) and its complementary sequence (left). The X axis represents a relative scale for distance along the chip surface, while the Y axis represents relative peak intensity. The right peak shows the attachment of the probe to the chip surface, and the left peak (slightly higher) demonstrates the binding of the complementary sequence to a surface-immobilized probe.

FIG. 11 is a schematic diagram illustrating the optical scanning of Probe 2 and its complementary sequence. The X axis represents a relative scale for distance along the chip surface, while the Y axis represents a relative peak intensity. The right peak shows the attachment of the probe to the surface, and the left peak (slightly higher) demonstrates the binding of the complementary sequence to the surface-attached probe.

FIG. 12 is an image of two chips. Four probe spots were placed on each chip: one chip for Probe 1 and one for Probe 2 Concentrated bacteria was resuspended in 1 ml (1:1) or 5 ml (1:5) PBS. The Probe 2 chip was rinsed with PBS, while the Probe 1 chip with dd H₂O, Sufficient bacteria remained on the probe 1 chip to allow naked-eye detection of bacteria following PBS rinse.

FIG. 13 is a computerized surface map showing the scanned surface over the E. coli section of Probe 1 chip, which was rinsed with dd H₂O after hybridization. The X and Z axes are relative distances on the chip surface, while the Y axis represents the intensities. The small peaks likely represent attached probe on the surface and some salt residue.

FIG. 14 is a computerized surface map showing the scanned surface over the Pseudomonas section of Probe 1 chip, which was rinsed with dd H₂O after hybridization. The X and Z axes are relative distances on the chip surface, while the Y axis represents the intensities. The large peak on the left demonstrates one spot. The peak on the right may be part of the other spot, but is more likely an artifact due to dust on the surface of the chip.

FIG. 15 is a computerized surface map showing the scanned surface of two spots for Probe 1 chip. The left side had fresh LB placed on Probe 1, while the right side had E. coli in fresh LB placed for hybridization. The peak intensities are not remarkable compared to the Pseudomonas data below. The X and Z axes are relative distances on the chip surface, while the Y axis represents the intensities.

FIG. 16 is a computerized surface map showing the scanned surface of two spots for Probe 2 chip. The left side had fresh LB placed on Probe 2, while the right side had Pseudomonas in fresh LB placed for hybridization. The peak intensities for this were very significant. Again, the X and Z axes are relative distances on the chip surface, while the Y axis represents the intensities. Similar results occurred for this experiment using Probe 1.

FIG. 17 is a diagram showing two dimensional optical images of scanned chips for Probe 2 (left) and Probe 1 (right). The cut off dilutions of 1/100,000 is evident, as peaks are noted for this dilution and do not exist for the 1/1×10⁶ dilution. The X axis represents relative distance on the chip, and the Y axis represents peak intensity.

FIG. 18 is a two dimensional map of an interferometric chip prepared using a single wavelength light source, with surface intensities representing detected P. aeruginosa.

### DETAILED DESCRIPTION OF THE INVENTION

A first aspect of the present invention relates to a method of detecting the presence of an otolaryngologic pathogen in a biological sample. This method involves providing a sensor device including (i) a substrate having two or more nucleic acid probes respectively confined to two or more distinct locations thereon, and (ii) a detector that detects the binding of target nucleic acids to the two or more nucleic acid probes, wherein a target nucleic acid is specific to one or more otolaryngologic pathogens; exposing a biological sample, or a portion thereof, to the sensor device under conditions effective to allow hybridization between the two or more nucleic acid probes and a target nucleic acid to occur, and detecting with the detector whether any target nucleic acid hybridizes to the two or more nucleic acid probes, where hybridization indicates the presence of the otolaryngologic pathogen in the biological sample and presence of more than one otolaryngologic pathogen can be detected simultaneously. The probes can be either bound to the surface of the substrate (e.g., in discrete locations) or the probes can be contained within vessels or reservoirs on the surface of the chip.

A second aspect of the present invention relates to a sensor device having a substrate to which has been bound two or more nucleic acid probes, and a detector that detects the hybridization of target nucleic acids to the two or more nucleic acid probes upon exposure to a biological sample, wherein a target nucleic acid is specific to one or more otolaryngologic pathogens and hybridization indicates presence of the otolaryngologic pathogen in the biological sample, the detector being capable of simultaneously detecting presence of more than one otolaryngologic pathogen in the biological sample.

A third aspect of the present invention relates to a sensor chip having a substrate to which has been bound two or more nucleic acid probes that will hybridize to a target nucleic acid of an otolaryngologic pathogen under conditions effective to allow hybridization, wherein the two or more probes are selected to hybridize, collectively, to target nucleic acids of two or more otolaryngologic pathogens.

Suitable sensor devices for use in the present invention include, without limitation, colorimetric nanocrystal sensors of the type disclosed in PCT International Application No. PCT/US02/18760 to Miller et al, filed Jun. 13, 2002 which is hereby incorporated by reference in its entirety; microcavity biosensors of the type disclosed in PCT International Application No. PCT/US02/05533 to Chan et al, filed Feb. 21, 2002, which is hereby incorporated by reference in its entirety; reflective interferometric sensors of the type disclosed in PCT International Application No. PCT/US02/34508 to Miller et al, filed Oct. 28, 2002, which is hereby incorporated by reference in its entirety; nucleic acid hairpin fluorescent sensors of the type disclosed in PCT International Application No. PCT/US2004/000093 to Miller et al, filed Jan. 2, 2004, which is hereby incorporated by reference in its entirety, and ricrofluidic sensor devices that utilize chips for carrying out hybridization using a fluorescently tagged probe or non-tagged probe, as described for example in PCT International Application No. PCT/US2004/015413 to Rothberg et al., filed May 17, 2004, which is hereby incorporated by reference in its entirety. 0

Colorimetric nanocrystal sensors can be used to detect the presence of one or more target nucleic acid molecules in a biological sample using fluorescence to indicate the presence of the target, as described in PCT International Application No. PCT/US02/18760 to Miller et al, filed Jun. 13, 2002. Although the cited application specifically excludes the use of nucleic acid probes, the use of nucleic acid probes is specifically contemplated in accordance with the present invention.

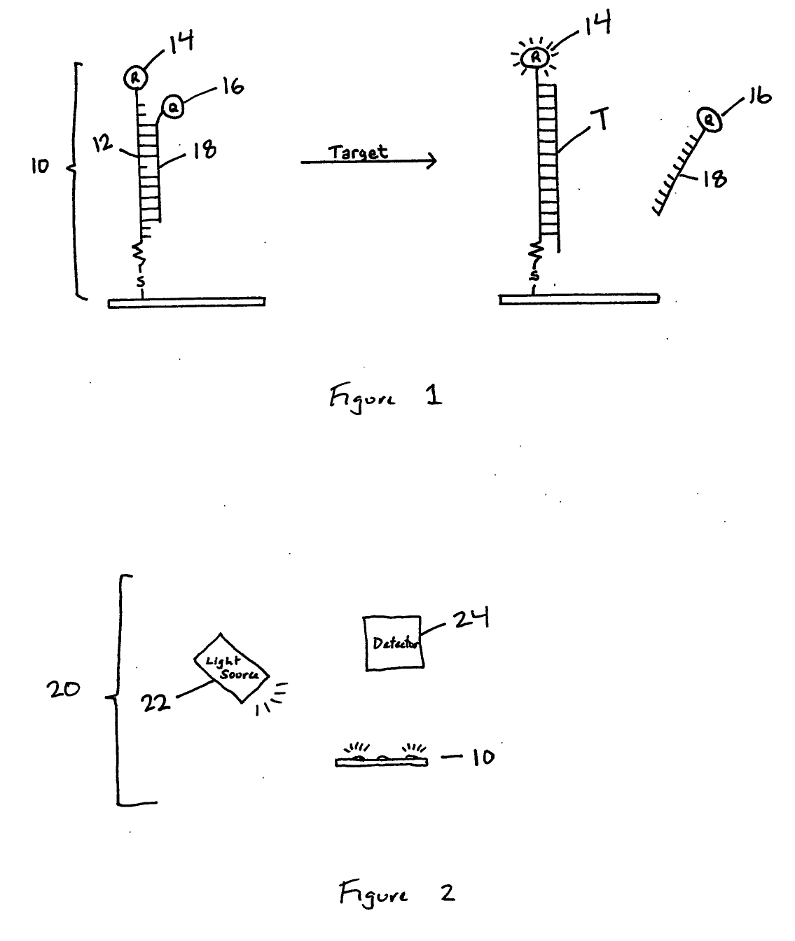

A shown in FIGS. 1 and 2, in a nanocrystal sensor chip 10 a nucleic acid probe 12 is attached to a nanocrystal particle 14. A quenching agent 16 that quenches, absorbs, or shifts fluorescence of the nanoparticle upon proximity to the nanoparticle is attached to a non-target nucleic acid sequence 18 that is complementary to a portion of the nucleic acid probe. In the absence of the target nucleic acid molecule, the non-target nucleic acid (tethered to the quenching agent) associates with the probe in such a way as to bring the quenching agent in close enough proximity to the nanoparticle to quench, absorb, or shift fluorescence of the nanoparticle. As shown in FIG. 1, in the presence of the target nucleic acid molecule T, which has a greater affinity for the probe than does the non-target nucleic acid, the non-target nucleic acid dissociates from the probe, thereby allowing the quenching agent to move out of proximity from the nanoparticle. A detector detects the change in fluorescence, which indicates the presence of the target in the sample.

To reduce its affinity for the nucleic acid probe, the non-target nucleic acid can contain a mismatch or other modification that would be apparent to one of ordinary skill in the art.

In at least one embodiment of the present invention the nanoparticle or the probe is also attached to an inert solid substrate. Multiple probe-nanoparticle complexes can be attached to the solid substrate and the substrate mapped according to probe, providing a way to identify the presence or absence of multiple targets in a single sample.

Suitable inert solid substrates according to this and other embodiments of this and all aspects of the present invention include, without limitation, silica and thin films of the type disclosed in PCT International Application No. PCT/US02/18760 to Miller et al, filed Jun. 13, 2002, which is hereby incorporated by reference in its entirety.

It should be apparent to one of ordinary skill in the art that nanocrystal chips in which neither the nanocrystal nor the probe is attached to a substrate can be employed using standard molecular beacons, or nanocrystal-derivatized beacons, in a solution-phase assay, as taught in PCT International Application No. PCT/2004/015413 to Rothberg et al., filed May 17, 2004, which is hereby incorporated by reference in its entirety.

The sensor chip is intended to be used as a component in a biological sensor device or system. Basically, as shown in FIG. 2, the sensor device 20 includes, in addition to the sensor chip 10, a light source 22 that illuminates the sensor chip at a wavelength suitable to induce fluorescent emissions by the nanoparticles, and a detector 24 positioned to capture any fluorescent emissions by the nanoparticles.

Suitable nanoparticles according to this and all aspects of the present invention can be designed using methods known in the art, including those disclosed in PCT International Application No. PCT/US02/18760 to Miller et al, filed Jun. 13, 2002 and PCT International Application No. PCT/US2004/000093 to Miller et al, filed Jan. 2, 2004.

Attaching of the various components of the nanocrystal sensor chip, including, without limitation, attaching the nanocrystal to the probe, the probe to the substrate, and the quenching agent to the non-target nucleic acid, can be achieved using methods known in the art, including those disclosed in PCT International Application No. PCT/US02/18760 to Miller et al, filed Jun. 13, 2002. Attachment of the various components includes, without limitation, direct attachment and attachment via a linker group, and combinations thereof, and disclosed in PCT International Application No. PCT/US02/18760 to Miller et al, filed Jun. 13, 2002. Regardless of the procedures employed, the nanocrystal particle and probe become bound or operably linked, and the nanocrystal or probe becomes bound or operably linked to the substrate. It is intended that the bond or fusion thus formed is the type of association which is sufficiently stable so that it is capable of withstanding the conditions or environments encountered during use thereof, i.e., in detection procedures. Preferably, the bond is a covalent bond, although other types of stable bonds can also be formed.

Suitable quenching agents and other fluorophores according to this and all aspect of the present invention can be designed using methods known in the art, including those disclosed in PCT International Application No. PCT/US02/18760 to Miller et al, filed Jun. 13, 2002. As used throughout herein, the terms “quenching agent” and “quenching substrate” include fluorophores that quench, absorb, or shift fluorescence of the respective nanoparticle, and combinations thereof. Exemplary quenching agents are metals, such as gold, platinum, silver, etc.

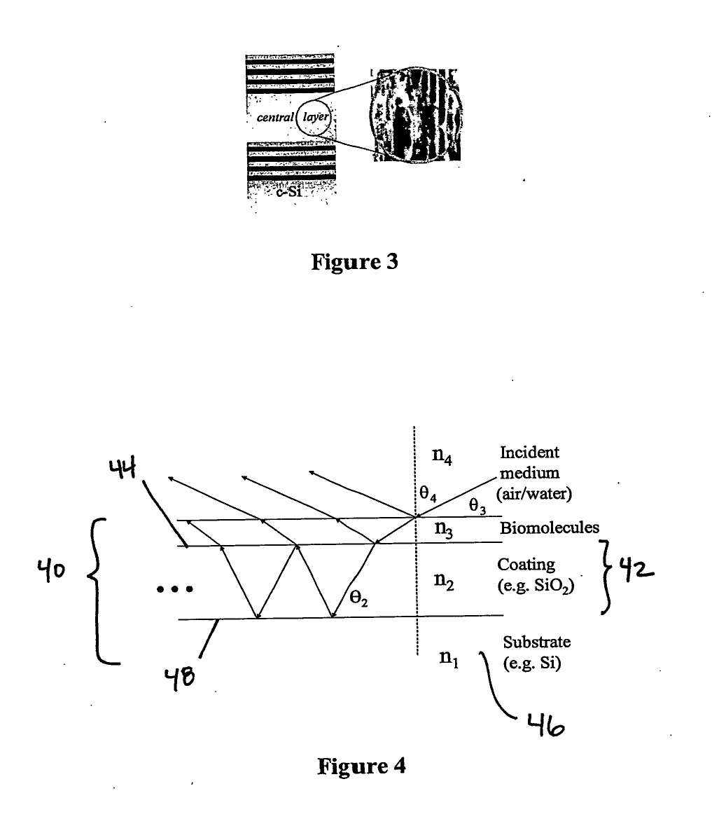

Microcavity biosensors can be used to detect the presence of one or more target nucleic acid molecules in a biological sample using the change in the refractive index to indicate the presence of the target, as described in PCT International Application No. PCT/US02/05533 to Chan et al, filed Feb. 21, 2002, which is hereby incorporated by reference in its entirety. Basically, a microcavity sensor chip includes two or more nucleic acid probes coupled to a porous semiconductor structure where a detectable change in refractive index occurs when a correlative target nucleic acid molecule becomes bound to one or more of the probes. The porous semiconductor structure has a configuration as illustrated in FIG. 3, with the upper layer and the lower layer on opposite sides of the central layer which is the microcavity.

The photoluminescent emission pattern of the sensor chip is measured. The structure is then exposed to a biological sample under conditions effective to allow binding of a target molecule in the sample to the one or more probes. The photoluminescent emission pattern is again measured and the first and second emission patterns are compared. The change in refractive index indicates the presence of the target in the sample. The semiconductor can be formed on any suitable semiconductor material, as disclosed in PCT International Application No. PCT/US02/05533 to Chan et al, filed Feb. 21, 2002, which is hereby incorporated by reference in its entirety.

Reflection of light at the top and bottom of the exemplary porous semiconductor structure results in an interference pattern that is related to the effective optical thickness of the structure. Binding of a target molecule to its corresponding probe, immobilized on the surfaces of the porous semiconductor structure, results in a change in refractive index of the structure and is detected as a corresponding shift in the interference pattern. The refractive index for the porous semiconductor structure in use is related to the index of the porous semiconductor structure and the index of the materials present (contents) in the pores. The index of refraction of the contents of the pores changes when the concentration of target species in the pores changes.

As shown in FIG. 2, the microcavity sensor chip of the present device is intended to be utilized as a component of a microcavity sensor device which also includes a source of illumination (e.g., argon, cadmium, helium, or nitrogen laser and accompanying optics) positioned to illuminate the microcavity sensor and a detector (e.g., collecting lenses, monochrometer, and detector) positioned to capture photoluminescent emissions from the microcavity sensor chip and to detect changes in photoluminescent emissions from the microcavity sensor chip. The source of illumination and the detector can both be present in a spectrometer. A computer with an appropriate microprocessor can be coupled to the detector to receive data from the spectrometer and analyze the data to compare the photoluminescence before and after exposure of the biological sensor to a target molecule.

Multiple target nucleic acid molecules can be detected with a single chip by arranging multiple probes on the same semiconductor structure. Multiple probes can include the same probes, different probes, or combinations thereof. The structure can be mapped to facilitate the detection of multiple targets as disclosed in PCT International Application No. PCT/US02/05533 to Chan et al, filed Feb. 21, 2002.

Suitable semiconductors and methods of forming the same include, without limitation, those disclosed in PCT International Application No. PCT/US02/05533 to Chan et al, filed Feb. 21, 2002.

Suitable methods of coupling the probes to the semiconductor are known in the art and include, without limitation, those described in PCT international Application No. PCT/US02/05533 to Chan et al, filed Feb. 21, 2002.

Reflective interferometric sensors can be used to detect the presence of one or more target nucleic acid molecules in a biological sample using reflective interference to indicate the presence of the target, as described in PCT International Application No. PCT/US02/34508 to Miller et al, filed Oct. 28, 2002.

One embodiment of an interferometric chip of the present invention is shown in FIG. 4. In this particular embodiment, the sensor chip 40 has a substrate 46 made of silicon with a coating 42 made of silicon dioxide on one surface, although other types of sensor chips made of other materials and layers can be used. The coating 42 contains front and back surfaces, the front surface 44 being presented to the media in which the sensor chip exists and the back surface 48 being in contact with the substrate 46. Nucleic acid probes (e.g. biomolecules) are attached to the coating.

It should be appreciated by those of ordinary skill in the art that any of a variety of substrates can be employed in the present invention.

The coating on the substrate is a reflective coating, that is, both the front and back surfaces of the coating are capable of reflecting incident light as illustrated in FIG. 4. The front and back face reflections result in destructive interference that can be measured.

A number of suitable coatings can be employed on the substrate. Silicon dioxide (glass) is a convenient coating because it can be grown very transparent and the binding chemistries are already worked out in many cases. Other transparent glasses and glass ceramics can also be employed. In addition, the coating can be a polymer layer or silicon nitride or an evaporated molecular layer. Coating procedures for application of such coatings onto substrates are well known in the art. It should also be appreciated that certain materials inherently contain a transparent oxidized coating thereon and, therefore, such receptor surfaces inherently include a suitable coating.

The coating of the sensor chip can be functionalized to include an nucleic acid probe that is specific for a desired target nucleic acid. In the embodiment illustrated in FIG. 4, the silicon dioxide coating on the surface of the receptor readily lends itself to modification to include thereon a nucleic acid probe (n3) that is receptive to adsorption of the one or more targets in the sample.

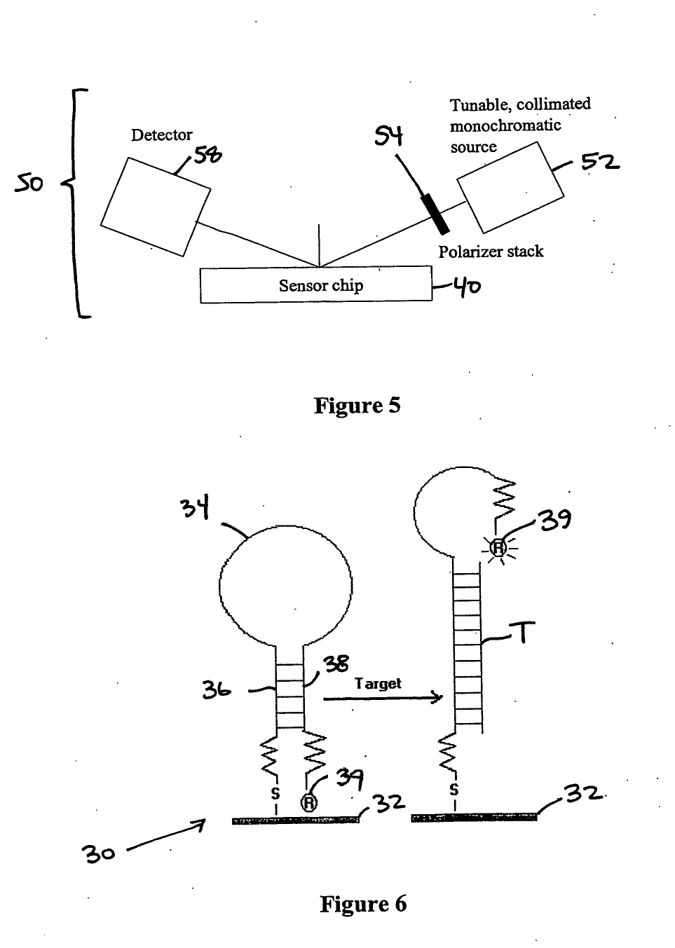

FIG. 5 illustrates an interferometric sensor device 50 in accordance with one embodiment of the present invention. The sensor device 50 includes a light source 52, a polarizer 54, a sensor chip 40, and a detector 54, although the sensor device can have other types and arrangements of components.

The light source 52 in the sensing system 20 generates and transmits a light at a set wavelength towards a surface of the sensor chip 40. In this particular embodiment the light source 52 is a tunable, collimated, monochromatic light source, although other types of light sources, such as a light source which is monochromatic, but not tunable or collimated could be used. A variety of different types of light sources, such as a light-emitting diode, a laser, or a lamp with a narrow bandpass filter, can be used. The medium in which the light travels from the light source 52 and polarizer 54 to the sensor chip 40 is air, although other types of mediums, such as an aqueous environment could be used.

The polarizer 54 is positioned in the path of the light from the light source 52 and polarizes the light in a single direction, although other arrangements for polarization are possible. Any of a variety of polarizers can be used to satisfactorily eliminate the p-component of the light from the light source 52. The polarizer 54 may also be connected to a rotational driving system, although other types of systems and arrangements for achieving this rotation can be used. Rotating the polarizer 54 (i.e. doing a full ellipsometric measurement) with the rotational driving system results in even better sensitivity of the system.

As an alternative to using a polarizer in addition to a non-polarized light source, a polarized light source can be utilized. A number of lasers are known to emit polarized light.

The detector 58 is positioned to measure the reflected light from the sensor chip 40.

Arraying as described in PCT International Application No. PCT/US02/34508 to Miller et al, filed Oct. 28, 2002, can be used to detect multiple target nucleic acid molecules.

Suitable substrates and coatings according to this and all aspects of the present invention include, without limitation, silicon oxide wafers carrying a thermal oxide coating, and translucent-coated substrates of the type disclosed in PCT International Application No. PCT/US02/34508 to Miller et al., filed Oct. 28, 2002, including without limitation, undoped silicon dioxide substrates coated with silicon dioxide.

Nucleic acid hairpin fluorescent sensors can be used to detect the presence of one or more target nucleic acid molecules in a biological sample using fluorescence to indicate the presence of the target, as described in PCT International Application No. PCT/US2004/000093 to Miller et al, filed Jan. 2, 2004.

As shown in FIG. 6, a nucleic acid hairpin fluorescent sensor chip 30 includes: a fluorescence quenching surface 32; two or more nucleic acid probes 34 each having first and second ends with the first end bound to the fluorescence quenching surface, a first region 36, and a second region 38 complementary to the first region; and a fluorophore 39 bound to the second end of the nucleic acid probe. Each probe has, under appropriate conditions, either a hairpin conformation with the first and second regions hybridized together, or a non-hairpin conformation.

While the probe remains in the hairpin conformation the fluorophore bound to the second end of the nucleic acid probe is brought into sufficiently close proximity to the fluorescence quenching surface such that the surface substantially quenches fluorescent emissions by the fluorophore. In contrast, while the probe remains in the non-hairpin conformation (i.e., when hybridized to a target), the fluorophore bound to the second end of the nucleic acid probe is no longer constrained in proximity to the fluorescence quenching surface. As a result of its physical displacement away from the quenching surface, fluorescent emissions by the fluorophore are substantially free of any quenching.

The sensor chip is intended to be used as a component in a biological sensor device or system. Basically, as shown in FIG. 2, the sensor device includes, in addition to the sensor chip, a light source that illuminates the sensor chip at a wavelength suitable to induce fluorescent emissions by the fluorophores associated with the probes bound to the chip, and a detector positioned to capture any fluorescent emissions by the fluorophores.

The sensor device containing a nucleic acid hairpin fluorescent chip with the probes in hairpin conformation is brought into contact with a biological sample under conditions effective to allow any target nucleic acid molecule in the sample to hybridize to the first and/or second regions of the nucleic acid probe(s) present on the sensor chip. Upon hybridization with a target, probes will assume a non-hairpin conformation, allowing the fluorophore bound to the probe to fluoresce and emission from the sensor becomes detectable. After contacting the sensor with the biological sample, the sensor chip is illuminated with light sufficient to cause emission of fluorescence by the fluorophores, and then it is determined whether or not the sensor chip emits detectable fluorescent emission. When fluorescent emission by a fluorophore is detected from the chip, that indicates that the nucleic acid probe is in the non-hairpin conformation and therefore that the target nucleic acid molecule is present in the sample.

The conditions under which the hairpin conformation exists are disclosed in PCT International Application No. PCT/US2004/000093 to Miller et al, filed Jan. 2, 2004. Suitable fluorescence quenching surfaces (e.g., gold, platinum, silver, etc.) and suitable fluorophores (e.g., dyes, proteins, nanocrystals, etc.) include, without limitation, those disclosed in PCT International Application No. PCT/US2004/000093 to Miller et al, filed Jan. 2, 2004. The nucleic acid probe can be bound to the fluorescent quenching surface and to the fluorophore using known methods including, without limitation, those described in PCT International Application No. PCT/US2004/000093 to Miller et al, filed Jan. 2, 2004.

Suitable substrates according to this and all aspects of the present invention include, without limitation, flourescence-quenching surfaces of the type disclosed in PCT International Application No. PCT/US2004/000093 to Miller et al, filed Jan. 2, 2004.

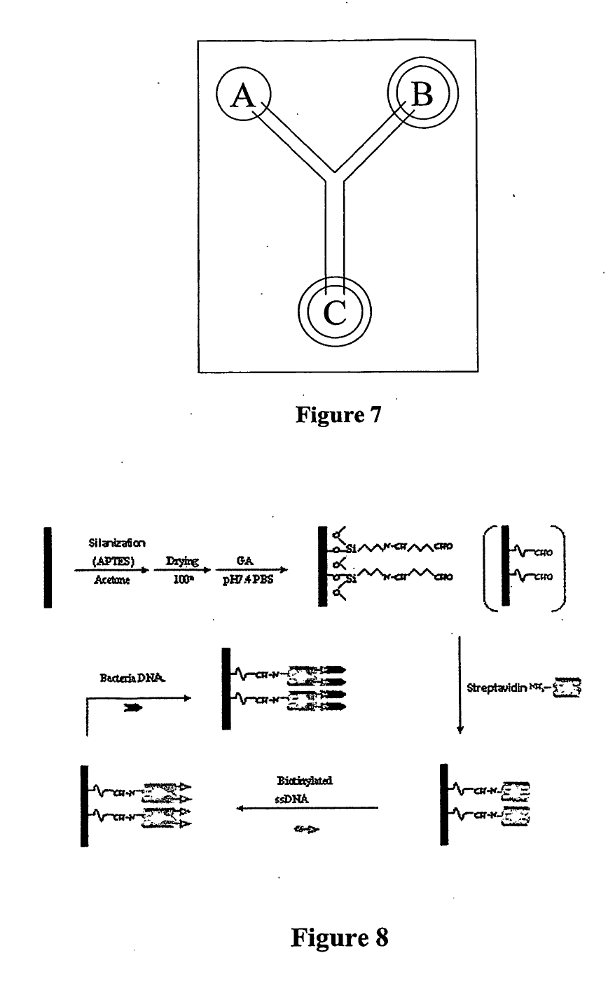

Microfluid sensors can be used to detect the presence of one or more target nucleic acid molecules in a biological sample using fluorescence to indicate the presence of the target, as described in PCT International Application No. PCT-US2004/015413 to Rothberg et al., filed May 17, 2004. A microfluidic chip, shown in FIG. 7, is constructed consisting of one reservoir (A) containing a solution of the quenched fluorescent probe, a fill port (B) into which the sample is introduced, and a visualization chamber (C), which can be probed with a spectrophotometer. The sample to be analyzed is introduced into (B), and then fluidic flow is induced to mix the contents of (A) and (B) in the channel, bringing the mixed solution to (C). If the target nucleic acid sequence is present, unquenching of the fluorescent probe occurs (or, alternatively, a color change occurs based on interaction/lack of interaction with Au nanoparticles), and the signal may be read spectrophotometrically through (C). It should be readily apparent to those skilled in the art that this scheme can be extended to a microfluidic chip incorporating several different probes, each occupying a separate reservoir, and able to be mixed independently with the sample in (B) using the addressable functions of the microfluidic chip.

Of the above embodiments, the interferometric sensor chip and device are preferred for practicing the present invention.

Suitable samples according to this and all aspects of the present invention can be either a tissue sample in solid form or in fluid form. The sample can also be present in an aqueous solution. Samples which can be examined include blood, water, a suspension of solids (e.g., food particles, soil particles, etc.) in an aqueous solution, or a cell suspension from a clinical isolate (such as a tissue homogenate from a mammalian patient).

Detection of the presence of the target in this and all aspects of the present invention can be achieved using conventional detection equipment appropriate for the type of sensor used, including, without limitation, fluorescence-detecting equipment disclosed in PCT International Application No. PCT/US02/18760 to Miller et al, filed Jun. 13, 2002, and PCT International Application No. PCT/US2004/000093 to Miller et al, filed Jan. 2, 2004, refractive index-detecting equipment of the type disclosed in PCT International Application No. PCT/US02/05533 to Chan et al, filed Feb. 21, 2002, and interference-detecting equipment of the type disclosed in PCT International Application No. PCT/US02/34508 to Miller et al, filed Oct. 28, 2002. Each of these references is hereby incorporated by reference in its entirety.

Suitable otolaryngologic pathogens according to this and all aspects of the present invention include, without limitation, Campylobacter jejuni, Campylobacter, Helicobater pylori, Listeria monocytogenes, Listeria, Staphylococcus aureus, Chlaniydia pneumoniae, Haemophilus influenzae, Streptococcus pneumoniae, α and β hemolytic Streptococcus, Streptococcus, Moraxella catarrhalis, Pseudomonas aeruginosa, Salmonella, viruses, including, without limitation, parainfluenzae viruses, influenzae viruses, and rhinoviruses, fungi, parasites, and prokaryotes.

Suitable nucleic acid probes according to this and all aspects of the present invention include, without limitation, those shown in Table 1, and combinations thereof. Other probes and combinations now known or hereinafter developed can also be used in the present invention. Any of these probe sequences can be converted for use in the hairpin scheme by adding self-complementary nucleotides to either end through methods that should be apparent to one of ordinary skill in the art. Suitable methods for converting sequences for use in the hairpin method include, without limitation, gene folding. By way of example, hairpin sequences can be formed by attaching the nucleic acid sequence CGCGACG- to the 5′ and 3′ ends of the nucleic acid probe. For example, SEQ ID NO: 1 would become SEQ ID NO: 23. In some cases that should be apparent to one of ordinary skill in the art, it may only be necessary to add CGACG- to each end, depending on the thermodynamic stability of the hairpin.

TABLE 1

Listing of Probe Sequences and Their
Target Organism

| Target | | |
|---|---|---|
| Organism | Probe Sequence | SEQ ID NO |
| Staphylococcus | acctataagactgggataactt | SEQ ID NO:1 |
| aureus | cgggaaac | |
| Staphylococcus | gacagcaagaccgtctttcact | SEQ ID NO:2 |
| aureus | tttgaacc | |
| Haemophilus | ctggggagtacggccgcaaggt | SEQ ID NO:3 |
| influenzae | taaaactc | |
| Haemophilus | gcgaaggcagccccttgggaat | SEQ ID NO:4 |
| influenzae | gtactgac | |
| Haemophilus | gcccttacgagtagggctacac | SEQ ID NO:5 |
| influenzae | acgtgcta | |
| Streptococcus | aaccacatgctccaccgcttgt | SEQ ID NO:6 |
| pneumoniae | gcgggccc | |
| Streptococcus | gtgcatggttgtcgtcagctcg | SEQ ID NO:7 |
| pneumoniae | tgtcgtga | |
| Moraxella | gggcgcaagctctcgctattag | SEQ ID NO:8 |
| catarrhalis | atgagcct | |
| Moraxella | ccatgccgcgtgtgtgaagaag | SEQ ID NO:9 |
| catarrhalis | gccttttg | |
| Chlamydia | acgatgcatacttgatgtggat | SEQ ID NO:10 |
| pneumoniae | ggtctcaa | |
| Chlamydia | ctcaaccccaagtcagcattta | SEQ ID NO:11 |
| pneumoniae | aaactatc | |
| Streptococcus | agtgcagaaggggagagtggaa | SEQ ID NO:12 |
| | ttccatgtgtagcggtgaaatg | |
| | cgtagatatatggagg | |
| Campylobacter | ccttacctgggcttgatatcct | SEQ ID NO:13 |
| jejuni or | aagaacct | |
| Campylobacter | | |
| Campylobacter | tcaccgcccgtcacaccatggg | SEQ ID NO:14 |
| jejuni or | agttgatt | |
| Campylobacter | | |
| Campylobacter | ggtataagccagcttaactgca | SEQ ID NO:15 |
| jejuni or | agacatac | |
| Campylobacter | | |
| Helicobacter | aagcagcaacgccgcgtggagg | SEQ ID NO:16 |
| pylori | atgaaggt | |
| Helicobacter | tatgctgagaactctaaggata | SEQ ID NO:17 |
| pylori | ctgcctcc | |
| Listeria | cggatttattgggcgtaaagcg | SEQ ID NO:18 |
| monocytogenes | cgcgcagg | |
| Listeria | cgaggtggagctaatcccataa | SEQ ID NO:19 |
| monocytogenes | aactattc | |
| Listeria | tcgtaaagtactgttgttagag | SEQ ID NO:20 |
| monocytogenes | aagaacaa | |
| Salmonella | agatgggattagcttgttggtg | SEQ ID NO:21 |
| | aggtaacg | |
| Salmonella | cggagggtgcaagcgttaatcg | SEQ ID NO:22 |
| | gaattact | |

Exemplary target nucleic acids include, without limitation, receptor molecules, preferably a biological receptor molecule such as a protein, RNA molecule, or DNA molecule. rRNA molecules are also suitable target nucleic acids, except to the extent the pathogen to be detected (i.e., a virus) does not contain ribosomes. In practice, the target nucleic acid is one which is associated with a particular disease state, a particular pathogen such as an otolaryngologic pathogen, etc. Such target nucleic acids, when identified in a sample, indicate the presence of a pathogen or the existence of a disease state (or potential disease state). These target nucleic acids can be detected from any source, including food samples, water samples, homogenized tissue from organisms, etc. Moreover, the biological sensor of the present invention can also be used effectively to detect multiple layers of biomolecular interactions, termed “cascade sensing.”

In this and all aspects of the present invention, the probes of a sensor chip can be specific to different nucleic acids, or to a combination of the same and different nucleic acids. Depending on the target nucleic acid, the target nucleic acid may be specific to one pathogen, or to more than one pathogen. Some target nucleic acids may, collectively, be specific to one pathogen. Chips can be designed using a combination of probe sequences that will identify the desired pathogens if present in a sample, as should be apparent to one of ordinary skill. Chips identifying pathogen species, genera, and other taxonomic groups can be designed in the same manner.

By exposing the sample to the probes, it is intended that a sufficient volume (e.g., 50-500 microliters, or more) of the sample can be manually or automatically applied to those locations on the chip where probes are retained, or to the entire chip. In the case of a microfluidic chip, the sample can be introduced to each vessel or channel.

Hybridization is carried out using standard techniques such as those described in Ausubel et al., Current Protocols in Molecular Biology, John Wiley & Sons, (1989). “High stringency” refers to DNA hybridization and wash conditions characterized by high temperature and low salt concentration, e.g., wash conditions of 650 C at a salt concentration of approximately 0.1×SSC. “Low” to “moderate” stringency refers to DNA hybridization and wash conditions characterized by low temperature and high salt concentration, e.g. wash conditions of less than 60 oC. at a salt concentration of at least 1.0×SSC. For example, high stringency conditions may include hybridization at about 42° C., and about 50% formamide; a first wash at about 65° C., about 2×SSC, and 1% SDS; followed by a second wash at about 65° C. and about 0.1×SSC. The precise conditions for any particular hybridization are left to those skilled in the art because there are variables involved in nucleic acid hybridizations beyond those of the specific nucleic acid molecules to be hybridized that affect the choice of hybridization conditions. These variables include: the substrate used for nucleic acid hybridization (e.g., charged vs. non-charged membrane); the detection method used; and the source and concentration of the nucleic acid involved in the hybridization. All of these variables are routinely taken into account by those skilled in the art prior to undertaking a nucleic acid hybridization procedure.

The present invention is useful for the diagnosis of ENT—(ear-nose-throat, or otolaryngologic) related infections. Otolaryngologic infections include, but are not limited to, middle ear infections, laryngeal infections, sinusitis, and throat infections. The specific organisms that can be targeted and identified with the ENT suite of chips include, but are not limited to, Campylobacter jejuni, Campylobacter, Helicobater pylori, Listeria monocytogenes, Listeria, Staphylococcus aureus, Chlamydia pneumoniae, Haemophilus influenzae, Streptococcus pneumoniae, α and β hemolytic Streptococcus, Streptococcus, Moraxella catarrhalis, Pseudomonas aeruginosa, Salmonella, otolaryngologic viruses like parainfluenzae, influenzae, and rhinovirus, and any host of fungi, parasites and prokaryotes contributing to diseases of the ear nose and throat.

The methods and devices disclosed herein are not limited to ENT related diseases and have potential applications in many other areas. This technology can be extended to include “organ specific” disease detection, which would consist of a chip designed for a specific disease state, and not explicitly a single organism. A few examples of these include, but are not limited to: Respiratory chips that detect pneumonia, bronchitis, and other pulmonary ailments from any host of viral, fungal, and bacterial pathogens. Gastrointestinal (GI) chips that can detect the presence of organisms causing diseases like ulcers, gastroenteritis, and small and large bowel infections from any host of bacterial, fungal, viral, and parasitic organisms. Wound chips that detect the presence if infections in wounds, including infections from implanted medical devices. Blood chips (sepsis chips) that detect the presence of bacteria, viruses, fungi, and parasites in blood. Neurologically focused chips that can be used to detect the presence of bacteria, viruses, and fungi in cerebrospinal fluid. Genitourinary chips that focus on a wide range of infections from urinary tract infections to sexually transmitted disease. General surveillance chips implanted in devices like respirators or used in health institutions to carry forth inspection of organisms common to nosocomial infections.

### EXAMPLES

The following examples are provided to illustrate embodiments of the present invention but are by no means intended to limit its scope.

### Example 1

### Preparation of Silicon Oxide Sensor Chips

Silicon oxide wafers 6″ diameter bearing a layer of 625-725 μm thick thermal oxide were obtained from a commercial vender (Xerox Corporation, Rochester N.Y.). These wafers were cut into 2.5×2.5 cm square chips. Care was taken to avoid scratching or otherwise marring the chip surface during all processing steps. All reagents (with the exception of DNA sequences, vide infra) were purchased from Sigma-Aldrich (St. Louis, Mo.) The chips were soaked in piranha etch solution (9 ml 3% H₂0₂ in 21 ml of 96% H₂SO₄) for 30 minutes. The chips were rinsed with ddH₂O and dried under a stream of nitrogen gas. The chips were then silanized with a 5% 3-aminopropyltrieethoxysilane solution 5% in acetone (96% reagent grade) for 1.5 hours. The chips were rinsed with ddH₂O and dried under a stream of nitrogen gas. After baling the silanized chips at 100 degrees C. for 1 hour, they were then treated with a solution of 2.5% Glutaraldehyde in 50 mM PBS (pH 7.4) for 45 minutes. The chips were rinsed with ddH₂O and dried under a stream of nitrogen gas. Each resulting glutaraldehyde-functionalized chip was then coated with 500 μl of streptavidin (0.05 mg/ml in PBS pH 7-7.5) for 45 minutes. The chips were rinsed with ddH₂O and dried under a stream of nitrogen gas. At this point, the chips were ready for the immobilization of the biotinylated DNA probes.

### Example 2

### Binding of Biotinylated DNA Probes to Silicon Oxide Sensor Chip

The well-studied streptavidin-biotin interaction (Wilchek et al., “Introduction to Avidin-Biotin Technology,” Methods Enzymol., 184:5-13 (1990)) was utilized to bind the DNA probes to the chip surface. Two biotinylated probes for Pseudomonas were purchased from a commercial supplier (Invitrogen Life Technologies, Carlsbad, Calif.) and used throughout this study:
Probe 1 5′-Biotin-CCT-TGC-GCT-ATC-AGA-TGA-GCC-TAG-GT-3′ (Knut et al., “Development and Evaluation of a 16S Ribosomal DNA Array-Based Approach for Describing Complex Microbial Communities in Ready-To-Eat Vegetable Salads Packed in a Modified Atmosphere,” Applied and Environmental Microbiology, 68:1146-1156 (2002), which is hereby incorporated by reference in its entirety)
Probe 2 5′-Biotin-CTG-AAT-CCA-GGA-GCA-3′ (Perry-O'Keefe et al., “Identification of Indicator Microorganisms Using Standardized PNA FISH Method,” Journal of Microbiological Methods, 47:281-292 (2001), which is hereby incorporated by reference in its entirety)

The biotinylated DNA probes were brought up to a concentration of 0.05 micromole/ml in PBS (pH 7.5). 5 μl of this solution was pipetted on the chips at each desired spot, and allowed to stand in a high-humidity chamber for 45 minutes. Chips were then rinsed with 50 mM PBS, followed by dd H₂O. The chips were now ready for treatment with either solutions of synthetic, complementary DNA, or with bacteria FIG. 8 shows a basic schematic of the chip functionalization process.

### Example 3

### Testing of Bound Probes

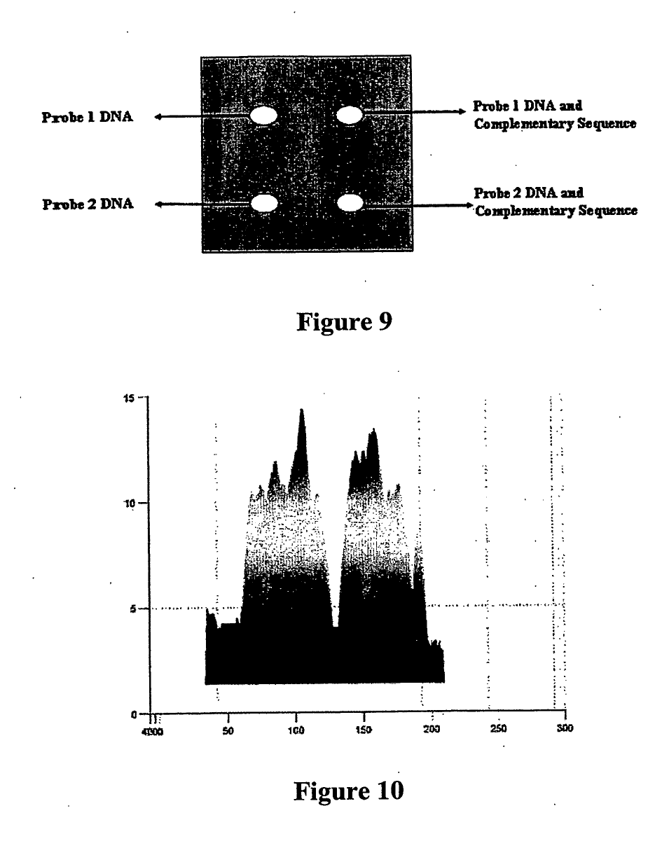

Complementary single stranded DNA sequences to Probe 1 and Probe 2 were purchased from a commercial supplier (Invitrogen Life Technologies, Carlsbad, Calif.), and diluted to a concentration of 0.01 micromole/ml in PBS. Each prepared chip's shape was traced onto graph paper, to mark the position placement of the probe and the subsequent complementary target sequence. The chips were prepared such that four spots were placed on the chip, with two having just placement of Probe 1 and Probe 2, and two having Probe 1 and Probe 2 with their complementary sequences, as shown in FIG. 9. Once the Probes had been placed, the chips were washed with dd H₂O and dried under a stream of nitrogen gas. Immediately thereafter, 0.05 μl of the target sequence was placed on the selected probes, and hybridization allowed to proceed at room temperature for 45 minutes. The chips were again washed with dd H₂O and dried with nitrogen gas. All chips were optically assessed within 24 hours of processing.

### Example 4

### Bacterial Processing Technique and Counts

Standard microbiology handling techniques were used to plate colonies and bring up culture solutions in LB media. The PAO-1 strain of Pseudomonas aeruginosa was obtained from the Department of Microbiology at Strong Memorial Hospital, and the JM109 strain of E. coli was obtained from a commercial supplier. Several colonies were swabbed from the culture plate into approximately 7-10 cc of LB media and cultured for 12 hours prior to experimentation. In the first set of experiments, 500 μl of cultured media was centrifuged at 12,000×G for 10 minutes. The pelleted cells were resuspended in 1 ml of 50 mM PBS (pH 7-7.5). In the first set of bacterial experiments, this solution was diluted 1:5 in PBS. For the second set of experiments, the bacteria were taken directly out of the liquid LB media after culture for chip experimentation. In the final serial dilution experiment, overnight cultures were taken and diluted in 0.9% NaCl in sequential 1/10 dilutions. Each dilution was then plated on LB agar plates in sets of 3, and the plates with 30-300 colonies were counted, with averages being obtained for the set dilution. Standard solution counts based on these dilutions were obtained using standard microbiology protocols for this procedure.

### Example 5

### Chip Bacterial Coating

Each chip was placed on grid paper, and the coordinates of the probes were marked. For each experiment, 5 μl of the bacterial preparation was placed on the coordinates of the probe and hybridized for 45 minutes at room temperature, followed by either a dd H₂O wash or a PBS wash and then nitrogen gas drying. To prevent spot drying, hybridization occurred in closed petri dishes with water soaked cotton balls to maintain moisture.

In the first set of bacteria experiments, the concentrated Pseudomonas and E. coli in 1:1 and 1:5 dilutions of PBS were spotted onto the Pseudomonas probes. The E. coli served as the control bacteria for each set of experiments. In the second set of experiments, 5 μl of fresh bacteria was taken from the LB media, and spotted on the Pseudomonas probes. Again, E. coli served as the control organism. LB media alone was also used as a control. In the last set of experiments, dilutions of Pseudomonas and E. coli in 0.9% NaCl were placed on the chips. These same dilutions were plated onto LB agarose plates for the counts. These chips were optically scanned to determine the detection limit for spot detection.

### Example 6

### Reflective Interferometry

All chips were processed by a single investigator in an established optics laboratory at the University of Rochester. The probe light for detection is derived from a 450 Watt Xe lamp monochromatized to approximately 1 nm bandwidth using a spectrometer. The light is guided through two apertures approximately 5 mm in diameter and separated by 60 mm to enforce collimation to better than 0.5 degrees. The beam is incident on the chip surface at 70.6 degrees, which is the reflectivity minimum. The reflected light is observed onto a Princeton Instruments (Monmouth, N.J.) CCD camera without imaging optics. In short, the peak intensity of the spots were compared to the background. The intensity of the peaks in the computer processed image are relative to the background intensities of non-spotted parts of the chip, and software automatically re-scales all the data for each chip. In the relevant Figures, the three dimensional X,Y,Z contour images and the one dimensional, X Y axis side-view of the three dimensional picture are shown for purposes of clarity.

### Example 7

### Preliminary Complementary Strand DNA Experiments

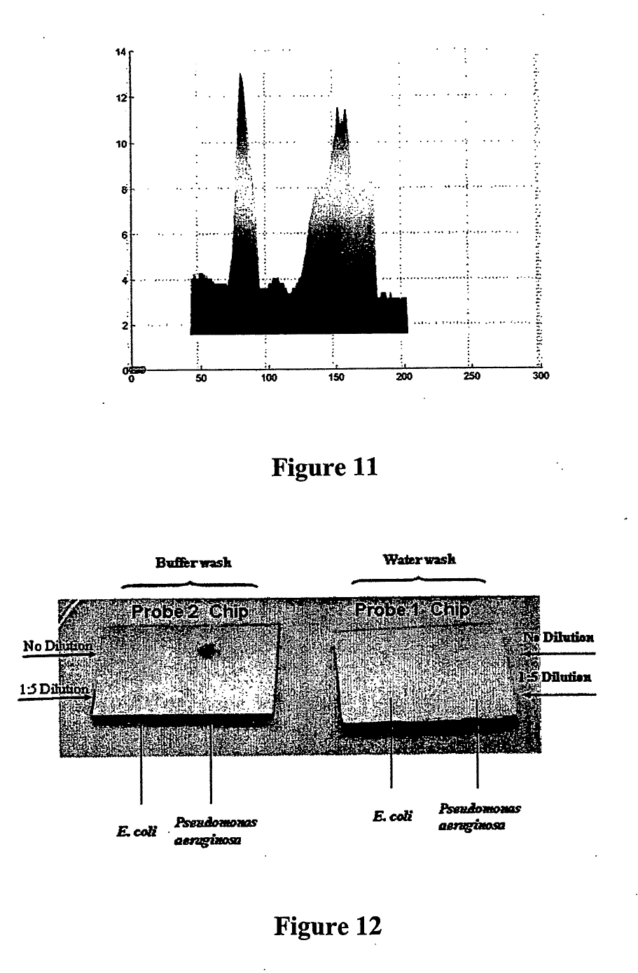

Probes 1 and 2 for Pseudomonas were optically evaluated with and without hybridization to the complementary sequence. The peak intensities were evaluated to assess visualization of this probe on the chip surface, and determine detection of the complementary sequence. The unhybridized probe sequence was placed in proximity to the probe and its complementary sequence, such that both could be visualized side-by-side. One dimensional views in FIG. 10 and FIG. 11 demonstrate the ability of the optical detection to see the probe and its differing intensity after binding its complementary sequence.

### Example 8

### Concentrated Bacteria

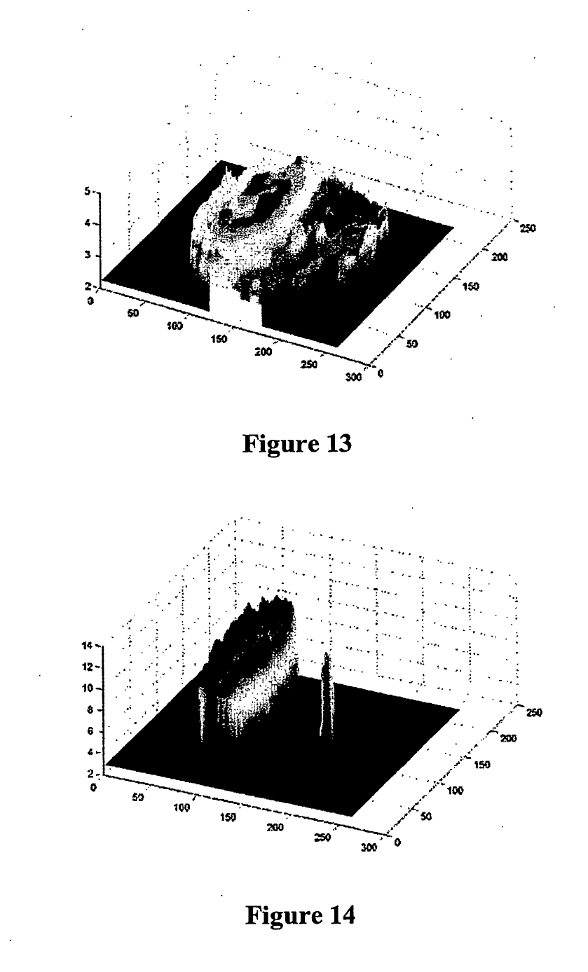

Following treatment with concentrated solutions of bacteria, the spots were immediately visible with the naked eye, without optical scanning (FIG. 10). This “naked-eye” detection is likely due to light scattering off the surface of the chip. After optically scanning the chips, large peaks were noted for both the 1:1 and the 1:5 dilutions of the concentrated Pseudomonas organisms after both the dd H₂O and PBS rinse, while the E. coli spots did not demonstrate comparable intensity peaks over background. The PBS rinse provides an obvious visual display of a “darker” spot, and this is reflected in the optical peak intensities. This is shown in FIG. 12. As described in Example 6, the current scanning technique and visualization algorithm makes a comparative display of the darkest spot on the chip to the background, and displays the relative intensities for that specific chip. Also visible was some salt streaking on the PBS rinsed chips after they are dried. The streak intensities were well below the spot intensities for these chips. FIGS. 13 and 14 are the scanned images over the E. coli and Pseudomonas sections, respectively, of Probe Chip 1. These figures show minimal binding to E. coli DNA but significant binding to Pseudomonas DNA.

### Example 9

### Fresh Bacteria

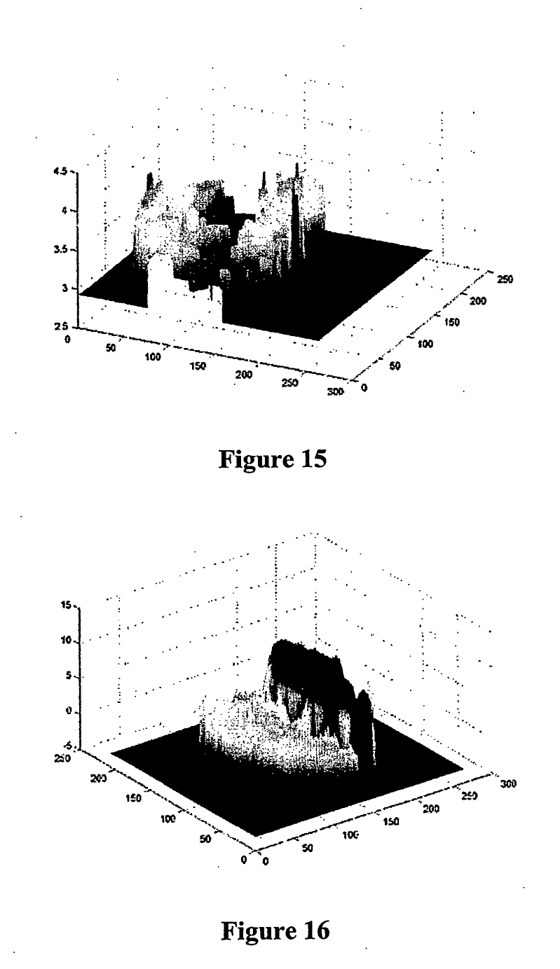

Four spots were placed on each chip, the top two with Probe 1 for Pseudomonas and the bottom 2 with Probe 2 for Pseudomonas. On each pair of two spots, fresh LB and fresh LB with cultured bacteria were placed on the probes. No recognizable peaks were noted for the control LB media alone. There were distinct peaks for the Pseudomonas in LB, and there were no peaks noted for E. coli in LB. FIGS. 15 and 16 are the scanned images of the Pseudomonas binding to Probes 1 and 2. The results for Probe 1 and Probe 2 were similar. All chips in this experiment were rinsed with PBS after hybridization to the probe.

### Example 10

### Bacterial Counts

The bacteria were diluted in 0.9% NaCl and spotted from this solution. These same dilutions were plated in sets of three, with hand counted colony averages of 30-300 being used for final counts. In the first set of bacterial counts, 2.49×10⁷ Colony Forming Units (CFU) of Pseudomonas were in each ml of solution. The dilution at which the peaks were no longer visible was 1/100,000, yielding a maximum optical detection of 24,900 CFU/ml of solution. The cut-off dilution was the same for chips using both Probe 1 and Probe 2. Since each spot consisted of only 5 μl of solution, the limit of detection was 125 CFU/spot detection. Repetition of this experiment was completed with limits of 160 CFU/5 μl spot being detected.

### Example 11

### Predicted Sequences Targeting Bacterial Pathogens

Database searches were carried out to predict selectivity for various pathogens. Should additional information be acquired in the future indicating that these sequences are not sufficiently selective, new probe sequences can be designed by one of ordinary skill in the art to carry out the methods disclosed herein.

It is expected that SEQ ID NO: 13, SEQ ID NO: 14, and SEQ ID NO: 15 can be used in tandem to identify Campylobacter jejuni. Alternatively, these sequences could be used to identify Campylobacter generally.

It is expected that either of SEQ ID NO: 16 and SEQ ID NO: 17 has selectivity for the Helicobater pylori 16S ribosome. Both can be used in combination to provide enhanced confidence in the detection method.

SEQ ID NO: 18, SEQ ID NO: 19, and SEQ ID NO: 20, used in combination, should provide absolute specificity for Listeria monocytogenes. Any one sequence used alone will identify Listeria, but may pick up more than one sub-species.

SEQ ID NO: 21 and SEQ ID NO: 22 primarily target Salmonella typhimurium, but will likely also pick up other Salmonella sub-species.

### Example 12

### Reflective Interferometry, Using Single Wavelength Light Source, for Detection of Pseudomonas aeroginosa

Detection may be accomplished using a single-wavelength reflective interferometry system. In this case, a silicon wafer with a thermal oxide layer of 141 nm was prepared, in order to provide a perfect null reflection condition for the illumination source. Immobilization of the probes occurred as described above; alternatively, amino-terminated DNA probes may be immobilized on epoxy-derivatized silicon chips, by analogy to methods disclosed in disclosed in PCT International Application No. PCT/US02/05533 to Chan et al., which is hereby incorporated by reference in its entirety. Visualization of the chip surface is accomplished using an apparatus as follows: the apparatus included a Melles Griot ImW helium-neon (HeNe) laser with a fixed wavelength of 632.5 nM. The beam passes through a lens aperture to collimate the beam followed by a polarizer and a HMS light beam chopper 221 frequency modulator set to 48.5 Hz. A 1 mm iris was placed in the path just before the chip to minimize beam elongation on the chip surface. A standard photodiode detector was used to collect the reflected beam and generate the electrical signal. The signal was then passed through a Stanford Research Systems SR570 Low-Noise preamp filter using positive bias voltage, 12 dB high-pass filter, 100 Hz filter frequency, 100 mA/V sensitivity and a −1 nA voltage offset. Once filtered, the signal is amplified with a Stanford Research Systems SR510 lock-in amplifier using 100 μV sensitivity, low dynamic resolution and a 300 ms time constant for data acquisition. Following filtering and amplification, the signal was processed via standard PC computer that is interfaced to the device via a National Instruments BNC 2010 connector block. The I/O signal generated by the connector block was input to the analysis software via a National Instruments PCI-6014 200 kS/s, 16-Bit, 16 analog input multifunction data acquisition system (DAQ) card within in a standard personal computer. Rastering of the entire chip surface was achieved by placing the prepared chip on a Vexta 2-phase stepping motor. The motor translated the chip in the XY dimensions and allows for a complete image of the chip surface to be obtained. Control of the XY stage and preliminary data analysis was carried out using the Lab View 7.0 environment (National Instruments) to control the position and speed of the stepper motor, receive data from the photodiode and map the position to the stepper motor, and displaying intensity as an X,Y pixel, with storage of the data in an Excel-readable file. Raw X,Y,Z (position, position, intensity) data was exported from this system, and imported as delimited text into Origin 7.0 for subsequent analysis. Analysis in Origin was carried out by transformation of the raw data into a regular [XYZ] matrix and mapping as a grayscale image. A modification of this apparatus replaced the XY stage with a fixed stage, and the photodiode and affiliated electronics with a CCD camera. The laser beam was expanded using standard optical methods to illuminate the region of the chip carrying the probe molecules.

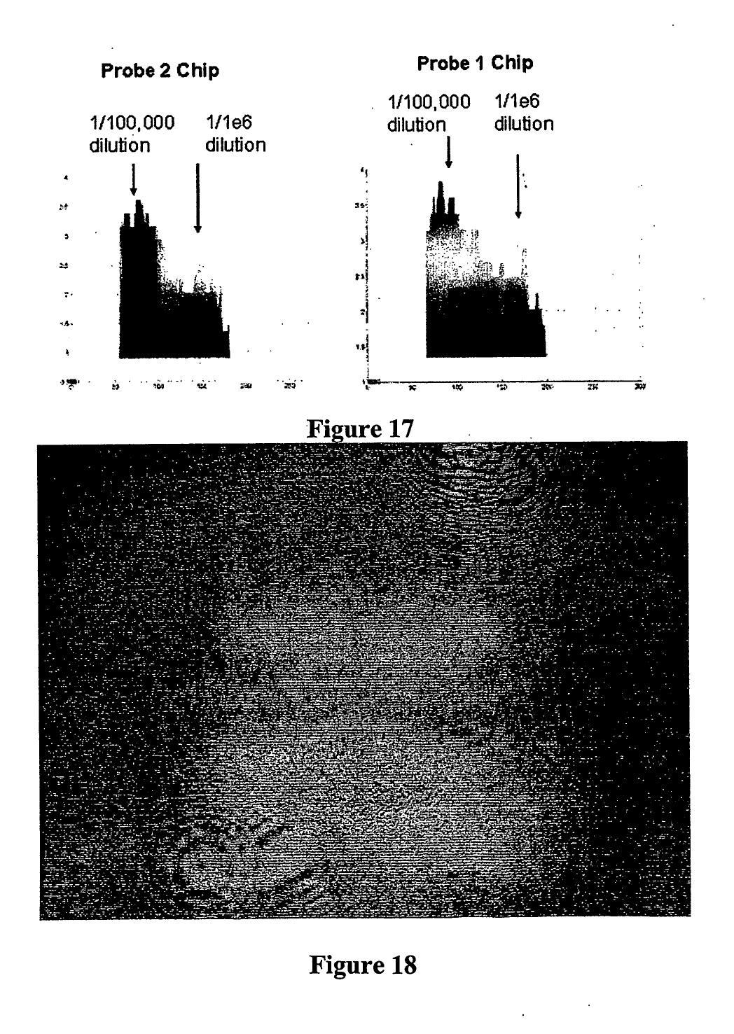

Pseudomonas cultures were grown overnight, spun down and the resuspend via 1 ml aliquots into PBS buffer. The resuspended cells were subject to freeze/thaw cycles to disrupt cellular membranes and sonicated to liberate DNA from the nuclei. The chip was prepared as described above, and then 200 microliters of the resulting sonicated culture was applied to the chip surface. Hybridization was allowed to occur for 1 hour. After washing with water, the chip was scanned with the above CCD-based system, resulting in the image shown in FIG. 18. Binding in two distinct locations is confirmed by the “bright spots”.

### Example 13

### Chip Functionalized with DNA Probe Sequences

It is predicted that chips could be functionalized with DNA probe sequences for detecting rRNA in bacteria, fungi, and parasites, as well as DNA or RNA of bacteria, fungi, viruses, and parasites. The target sequences are not necessarily limited to rRNA

Multiple probes could be arrayed on a single chip for point of care detection. These probes can be for organ-specific disease combinations (like a chip for all sinus infections), combining probes for bacteria, viruses, or fungi. They can also be for disease specific combinations (URI viral chip, bacterial pharyngitis chip, fungal otitis chip), etc.

Single probes could be placed on chips for rapid point of care detection. An example would be a new rapid streptococcus point of care chip.

### Example 14

### Antibody-Functionalized Chip

It is predicted that chips could be functionalized with antibodies for detection of bacteria, viruses, fungi, or any host of allergic diseases. These antibodies would be raised towards specific protein, peptide, or small molecule targets, unique to the organism or disease of interest like allergic rhinitis. Patient serum or secretions could be placed on these chips. The diagnosis would be generated using these antibody mobilized chips.

### Example 15

### Biomarker Chip

It is predicted that chips could be functionalized with DNA or antibodies for rapid molecular detection of cellular morphology. These biomarker chips would allow for rapid detection of cellular features, as in determining prognostic factors for cancer behavior. Examples of such biomarkers include, but are not limited to, p53, Bcl-2, Cyclin D1, c-myc, p21ras, c-erb B2, and CK-19.

### Example 16

### Hyaluronic Acid Disaccharide Chip

It is predicted that chips could be functionalized with hyaluronic acid disaccharide for the detection of Streptococcus pneumoniae hyaluronate lyase. This chip could be used to identify presence of the most common etiologic agent responsible for AOM (acute otitis media) and for invasive bacterial infections in children of all age groups.

### Example 17

### Pepsin Activity Detection Chip

It is predicted that chips could be functionalized with proteins or peptides that indicate presence of pepsin through the inherent enzymatic activity and in turn identify possible acid reflux disease (GERD). This would be enabled through the use of proteins or peptides that are the normal substrates of pepsin enzymatic activity

### Example 18

### Chip for Diagnosis of Cerebrospinal Fluid Leaks

It is predicted that a chip could be designed to rapidly detect molecules like B-2 transferrin that are sensitive to the diagnosis of cerebrospinal fluid leaks. These chips could use any range of protein detection techniques to detect the presence of this molecule in patient sinus or ear specimens.

### Example 19

### Lipopolysaccharide a Detection Chip

It is predicted that chips could be designed to detect Lipopolysaccharide A (LPS). This could be done by immobilizing molecules on the surface of the chip that are sensitive and specific for the molecule LPS, the causative agent behind most cases of sepsis.

### Example 20

### Methods of Use

Predictably, chips could be stored in the physician's office, hospital, or operating room suite, wherever point of care detection is most convenient for the physician or other health care practitioner. These chips could also be used by clinical laboratories to make more accurate and more rapid detection.

For infectious diseases, there are three predicted methods for sample collection in the diseased organ system. First, upon suspicion of an infectious disease etiology, the infection site would be swabbed as per usual protocol for obtaining cultures for microbiological processing. The practitioner may or may not see clinical evidence of the infection. Given the chip sensitivity, an area could be swabbed if the practitioner has the mere suspicion of infection. Second, for other diseases like sinusitis or urinary tract infections, the patient may produce a sample (sputum, urine, etc) that can be collected for chip evaluation. Third, for diseases like sepsis or meningitis, appropriate serum or CSF could be collected by a licensed practitioner and placed on the chip.

For other categories of diagnostic detection not related to infectious etiologies, similar techniques could be employed to obtain a patient sample and place it on the chip for functionalization and detection.

Once the sample is collected, it would be placed on the appropriate chip for diagnosis. As noted above, the chip may be designed per disease organ, per infectious etiology, as a single organisms detection tool, or for any group of relevant molecules necessitating detection. Once the sample is placed on the chip, it would be processed potentially through a series of simple washes. It is anticipated that with continued technology development, multiple washes will not be needed. The chip would then be scanned in the examination setting. This detection device would use a laser to first scan the surface of the chip. On multiple probe chips, there would be a recorded map of the probes such that specific target binding can be assessed. The laser would reflect onto a photodiode, and a computer processor would determine positive binding based on previous set algorithms.

The scanned chip data would translate into a simple report of infectious etiology for the physician/health practitioner to evaluate. This data could then be used to determine treatment options for the patient.

One alternative technique for this device would be a delayed evaluation after the swabbed sample is incubated for several hours and then wiped onto the chip. This would still allow for point of care detection, or it may be an alternative to current clinical laboratory organism evaluation techniques.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A method of detecting presence of an otolaryngologic pathogen in a biological sample:
providing a sensor device comprising (i) a substrate having two or more nucleic acid probes respectively confined to two or more distinct locations thereon, and (ii) a detector that detects the binding of target nucleic acids of a biological sample to the two or more nucleic acid probes, wherein a target nucleic acid is specific to one or more otolaryngologic pathogens;
exposing the biological sample, or a portion thereof, to the sensor device under conditions effective to allow hybridization between the two or more nucleic acid probes and a target nucleic acid to occur; and
detecting with the detector whether any target nucleic acid hybridizes to the two or more nucleic acid probes, wherein hybridization indicates presence of the otolaryngologic pathogen in the biological sample and presence of more than one otolaryngologic pathogen can be detected simultaneously.

2. The method according to claim 1 wherein the otolaryngologic pathogen is selected from the group of Campylobacter jejuni, Campylobacter, Helicobater pylori, Listeria monocytogenes, Listeria, Staphylococcus aureus, Chlamydia pneumoniae, Haemophilus influenzae, Streptococcus pneumoniae, α and β hemolytic Streptococcus, Streptococcus, Moraxella catarrhalis, Pseudomonas aeruginosa, Salmonella, parainfluenzae viruses, influenzae viruses, rhinoviruses, otolaryngologic fungi, otolaryngologic parasites, otolaryngologic parasites, and otolaryngologic prokaryotes.

3. The method according to claim 1 wherein the target nucleic acid is a DNA molecule.

4. The method according to claim 1 wherein the target nucleic acid is an RNA molecule.

5. The method according to claim 1 wherein the target nucleic acid is an rRNA molecule.

6. The method according to claim 1 wherein the two or more nucleic acid probes are coupled to the substrate.

7. The method according to claim 6 wherein the substrate comprises a silicon oxide wafer carrying a thermal oxide coating.

8. The method according to claim 6 wherein the sensor device further comprises one or more nanocrystal particles comprising a semiconductor material, the one or more nanocrystal particles being coupled to the substrate via the two or more nucleic acid probes.

9. The method according to claim 8 wherein the sensor device further comprises one or more quenching agents each coupled to a non-target nucleic acid, the non-target nucleic acid being reversibly coupled to a nucleic acid probe with an affinity that is lower than the affinity between the nucleic acid probe and the target nucleic acid.

10. The method according to claim 8 wherein said detecting comprises:
illuminating the sample and sensor device; and
measuring fluorescence by the one or more nanocrystal particles, whereby fluorescence indicates displacement of the non-target nucleic acid and quenching agent from the nucleic acid probe.

11. The method according to claim 6 wherein the substrate comprises a porous semiconductor structure comprising a central layer interposed between upper and lower layers, each of the upper and lower layers including strata of alternating porosity.

12. The method according to claim 11 wherein said detecting comprises measuring the refractive index of the substrate, whereby a change in the refractive index indicates the binding of a target nucleic acid to a probe.

13. The method according to claim 6 wherein the substrate includes a translucent coating having front and back surfaces and the detector comprises a light source positioned to illuminate the substrate whereby, in the absence of a target nucleic acid, near perfect interference occurs between light reflected by the front and back surfaces.

14. The method according to claim 13 wherein said detecting comprises measuring the light reflected by the front and back surfaces of the coating, whereby loss of interference indicates binding of a target nucleic acid to a probe.

15. The method according to claim 13 wherein the substrate comprises undoped silicon and the coating comprises silicon dioxide.

16. The method according to claim 6 wherein the substrate comprises a fluorescence quenching surface and each of the two or more probes comprises first and second ends with the first end bound to the fluorescence quenching surface and the second end bound to a fluorophore, a first region, and a second region complementary to the first region, the probe having, under appropriate conditions, either a hairpin conformation with the first and second regions hybridized together or a non-hairpin conformation, whereby when the probe is in the hairpin conformation, the fluorescence quenching surface substantially quenches fluorescent emissions by the fluorophore, and when the probe is in the non-hairpin conformation fluorescent emissions by the fluorophore are substantially free of quenching by the fluorescence quenching surface.

17. The method according to claim 16 wherein said detecting comprises:
illuminating the sample and sensor device; and
measuring fluorescence by the fluorophore, whereby fluorescence indicates that at least one of the two or more probes is in the non-hairpin conformation.

18. The method according to claim 1 wherein the two or more nucleic acid probes are each retained within a separate microfluid vessel or channel.

19. The method according to claim 18 wherein the substrate is in the form of a microfluid chip comprising a plurality of microfluid vessels and channels.

20. The method according to claim 19 wherein said detecting comprises
exposing a plurality of metal nanoparticles to the biological sample and the two or more nucleic acid probes, and
determining whether a color change occurs after said exposing the plurality of metal nanoparticles, whereby a color change indicates substantial aggregation of the plurality of metal nanoparticles in the presence of the target nucleic acid.

21. The method according to claim 1 wherein the two or more probes comprise the nucleotide sequence selected from the group of SEQ ID NO: 1, SEQ ID NO: 2, SEQ ID NO:3, SEQ ID NO: 4, SEQ ID NO:5, SEQ ID NO:6, SEQ ID NO:7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ BD NO: 20, SEQ ID NO: 21, SEQ ID NO: 22, and complements thereof; and combinations thereof.

22. The method according to claim 1 wherein the two or more probes are specific to different otolaryngologic pathogens.

23. The method according to claim 1 wherein at least two of the two or more probes are specific to the same otolaryngologic pathogen and at least one additional probe is specific to a different otolaryngologic pathogen.

24. A sensor device comprising:
a substrate having two or more nucleic acid probes respectively confined to two or more distinct locations thereon and
a detector that detects the hybridization of target nucleic acids to the two or more nucleic acid probes upon exposure to a biological sample, wherein a target nucleic acid is specific to one or more otolaryngologic pathogens and hybridization indicates presence of the otolaryngologic pathogen in the biological sample, the detector being capable of simultaneously detecting presence of more than one otolaryngologic pathogen in the biological sample.

25. The sensor device according to claim 24 wherein the otolaryngologic pathogen is selected from the group of Campylobacter jejuni, Campylobacter, Helicobater pylori, Listeria monocytogenes, Listeria, Staphylococcus aureus, Chlamydia pneumoniae, Haemophilus influenzae, Streptococcus pneumoniae, α and β hemolytic Streptococcus, Streptococcus, Moraxella catarrhalis, Pseudomonas aeruginosa, Salmonella, parainfluenzae viruses, influenzae viruses, rhinoviruses, otolaryngologic fungi, otolaryngologic parasites, otolaryngologic parasites, and otolaryngologic prokaryotes.

26. The sensor device according to claim 24 wherein the two or more nucleic acid probes are coupled to the substrate.

27. The sensor device according to claim 26 wherein the substrate comprises a silicon oxide wafer carrying a thermal oxide coating.

28. The sensor device according to claim 26 wherein the sensor device further comprises one or more nanocrystal particles comprising a semiconductor material, the one or more nanocrystal particles being attached to the substrate via the two or more nucleic acid probes.

29. The sensor device according to claim 26 wherein the sensor device further comprises one or more quenching agents each coupled to a non-target nucleic acid, the non-target nucleic acid being reversibly coupled to a nucleic acid probe with an affinity that is lower than the affinity between the nucleic acid probe and the target nucleic acid.

30. The sensor device according to claim 26 wherein the substrate comprises a porous semiconductor structure comprising a central layer interposed between upper and lower layers, each of the upper and lower layers including strata of alternating porosity.

31. The sensor device according to claim 26 wherein the substrate includes a translucent coating having front and back surfaces and the detector comprises a light source positioned to illuminate the substrate whereby, in the absence of target nucleic acid, near perfect interference occurs between light reflected by the front and back surfaces.

32. The sensor device according to claim 31 wherein the substrate comprises undoped silicon and the coating comprises silicon dioxide.

33. The sensor device according to claim 26 wherein the substrate comprises a fluorescence quenching surface and each of the two or more probes comprises first and second ends with the first end bound to the fluorescence quenching surface and the second end bound to a fluorophore, a first region, and a second region complementary to the first region, the probe having, under appropriate conditions, either a hairpin conformation with the first and second regions hybridized together or a non-hairpin conformation, whereby when the probe is in the hairpin conformation, the fluorescence quenching surface substantially quenches fluorescent emissions by the fluorophore, and when the probe is in the non-hairpin conformation fluorescent emissions by the fluorophore are substantially free of quenching by the fluorescence quenching surface.

34. The method according to claim 24 wherein the two or more nucleic acid probes are each retained within a separate microfluid vessel or channel.

35. The method according to claim 18 wherein the substrate is in the form of a microfluid chip comprising a plurality of microfluid vessels or channels.

36. The sensor device according to claim 24 wherein the two or more nucleic acid probes comprise the nucleotide sequence selected from the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and complements thereof; and combinations thereof.

37. The sensor device according to claim 24 wherein the two or more probes are specific to different otolaryngologic pathogens.

38. The sensor device according to claim 24 wherein at least two of the two or more probes are specific to the same otolaryngologic pathogen and at least one additional probe of the two or more probes is specific to a different otolaryngologic pathogen.

39. A sensor chip comprising a substrate having two or more nucleic acid probes respectively confined to two or more distinct locations thereon, the nucleic acid probes hybridizing to a target nucleic acid of an otolaryngologic pathogen under suitable hybridization conditions, wherein the two or more probes are selected to hybridize, collectively, to target nucleic acids of two or more otolaryngologic pathogens.

40. The sensor chip according to claim 39 wherein the otolaryngologic pathogen is selected from the group of Campylobacter jejuni, Campylobacter, Helicobater pylori, Listeria monocytogenes, Listeria, Staphylococcus aureus, Chlamydia pneumoniae, Haemophilus influenzae, Streptococcus pneumoniae, α and β hemolytic Streptococcus, Streptococcus, Moraxella catarrhalis, Pseudomonas aeruginosa, Salmonella, parainfluenzae viruses, influenzae viruses, rhinoviruses, otolaryngologic fungi, otolaryngologic parasites, otolaryngologic parasites, and otolaryngologic prokaryotes.

41. The sensor chip according to claim 39 wherein the two or more nucleic acid probes are coupled to the substrate.

42. The sensor chip according to claim 41 wherein the substrate comprises a silicon oxide wafer carrying a thermal oxide coating.

43. The sensor chip according to claim 41 wherein the sensor chip further comprises one or more nanocrystal particles comprising a semiconductor material, the one or more nanocrystal particles being attached to the substrate via the two or more nucleic acid probes.

44. The sensor chip according to claim 41 wherein the sensor chip further comprises one or more quenching agents each coupled to a non-target nucleic acid, the non-target nucleic acid being reversibly coupled to a nucleic acid probe with an affinity that is lower than the affinity between the nucleic acid probe and the target nucleic acid.

45. The sensor chip according to claim 41 wherein the substrate comprises a porous semiconductor structure comprising a central layer interposed between upper and lower layers, each of the upper and lower layers including strata of alternating porosity.

46. The sensor chip according to claim 41 wherein the substrate includes a translucent coating having front and back surfaces and the detector comprises a light source positioned to illuminate the substrate whereby, in the absence of target nucleic acid, near perfect interference occurs between light reflected by the front and back surfaces.

47. The sensor chip according to claim 46 wherein the substrate comprises undoped silicon and the coating comprises silicon dioxide.

48. The sensor chip according to claim 41 wherein the substrate comprises a fluorescence quenching surface and each of the two or more probes comprises first and second ends with the first end bound to the fluorescence quenching surface and the second end bound to a fluorophore, a first region, and a second region complementary to the first region, the probe having, under appropriate conditions, either a hairpin conformation with the first and second regions hybridized together or a non-hairpin conformation, whereby when the probe is in the hairpin conformation, the fluorescence quenching surface substantially quenches fluorescent emissions by the fluorophore, and when the probe is in the non-hairpin conformation fluorescent emissions by the fluorophore are substantially free of quenching by the fluorescence quenching surface.

49. The method according to claim 39 wherein said the two or more nucleic acid probes are each retained within a separate microfluid vessel or channel.

50. The method according to claim 48 wherein the substrate is in the form of a microfluid chip comprising a plurality of microfluid vessels and channels.

51. The sensor chip according to claim 39 wherein the one or more probes comprise a nucleotide sequence selected from the group of SEQ ID NO: 1, SEQ ID NO:2, SEQ ID NO:3, SEQ ID NO:4, SEQ ID NO: 5, SEQ ID NO:6, SEQ ID NO:7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and complements thereof; and combinations thereof.

52. The sensor chip according to claim 39 wherein the two or more probes are specific to different otolaryngologic pathogens.

53. The sensor chip according to claim 39 wherein at least two of the two or more probes are specific to the same otolaryngologic pathogen and at least one additional probe of the two or more probes is specific to a different otolaryngologic pathogen.

54. A nucleic acid probe comprising a nucleic acid sequence selected from the group of:
SEQ ID NO: 1, SEQ ID NO: 2, SEQ ID NO:3, SEQ ID NO: 4, SEQ ID NO:5, SEQ ID NO:6, SEQ ID NO:7, SEQ ID NO: 8, SEQ ID NO:9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, and complements thereof; and combinations thereof.

55. The nucleic acid probe of claim 53 further comprising a fluorophore conjugated to the nucleic acid probe.

56. The nucleic acid probe of claim 53 wherein the nucleic acid probe is capable of self-hybridizing to form a hairpin structure.
